(19) United States
(12) Patent Application Publication
(10) Pub. No.: US 2010/0311084 A1
(43) Pub. Date: Dec. 9, 2010

(54) METHODS FOR PREDICTING A PATIENT'S RESPONSE TO EGFR INHIBITORS

(75) Inventors: Stacey Brower, Pittsburgh, PA (US); Shara D. Rice, Pittsburgh, PA (US); Theresa R. Cassino, Pittsburgh, PA (US); Lahari Sakhamuri, Pittsburgh, PA (US); Heather M. Buechel, Pittsburgh, PA (US); Lauren M. Hancher, Pittsburgh, PA (US)

Correspondence Address: COOLEY LLP;ATTN: Patent Group, Suite 1100, 777 - 6th Street, NW, WASHINGTON, DC, 20001, US

(73) Assignee: PRECISION THERAPEUTICS, INC., Pittsburgh, PA (US)

(21) Appl. No.: 12/795,286

(22) Filed: Jun. 7, 2010

Related U.S. Application Data
(63) Continuation-in-part of application No. 12/466,129, filed on May 14, 2009.
(60) Provisional application No. 61/142,809, filed on Jan. 6, 2009.
(60) Provisional application No. 61/053,094, filed on May 14, 2008.

(51) Int. Cl. G01N 33/574

(52) U.S. Cl. 435/7.23

(57) ABSTRACT

The present invention provides methods for individualizing chemotherapy for cancer treatment, and particularly for evaluating a patient's responsiveness to one or more epidermal growth factor receptor (EGFR) inhibitors prior to treatment with such agents. Particularly, the invention provides an in vitro chemoresponse assay for predicting a patient's response to a monoclonal EGFR antibody, such as cetuximab. The method generally comprises culturing malignant cells from a patient's specimen (e.g., biopsy specimen), contacting the cultured cells with a monoclonal EGFR antibody that is a candidate treatment for the patient, and evaluating the cultured cells for a response to the drug. In certain embodiments, monolayer(s) of malignant cells are cultured from explants prepared by mincing tumor tissue, and the cells of the monolayer are suspended and plated for chemosenstivity testing. The in vitro response to the drug as determined by the method of the invention is correlative with the patient's in vivo response upon receiving the monoclonal EGFR antibody during chemotherapeutic treatment (e.g., in combination with other standardized or individualized chemotherapeutic regimen).

### PRIORITY

This application is a Continuation-in-Part of U.S. application Ser. No. 12/466,129 filed May 14, 2009, which claims priority to U.S. Provisional Application 61/142,809 filed Jan. 6, 2009 and U.S. Provisional Application No. 61/053,094 filed May 14, 2008, each of which is hereby incorporated by reference in its entirety and for all purposes.

### FIELD OF THE INVENTION

The present invention relates to individualizing cancer treatment, and particularly to individualizing cancer treatment by evaluating a patient for responsiveness to an EGFR inhibitor prior to therapy with such agent.

### BACKGROUND

Epidermal growth factor receptor (EGFR) inhibitors have been approved or tested for treatment of a variety of cancers, including non-small cell lung cancer (NSCLC), head and neck cancer, colorectal carcinoma, and Her2-positive breast cancer, and are increasingly being added to standard therapy. EGFR inhibitors, which may target either the intracellular tyrosine kinase domain or the extracellular domain of the EGFR target, are generally plagued by low population response rates, leading to ineffective or non-optimal chemotherapy in many instances, as well as unnecessary drug toxicity and expense. For example, a reported clinical response rate for treatment of breast carcinoma with lapatinib (a small molecule EGFR tyrosine kinase inhibitor) is about 10% [New England J. Med. 2006; 355:2733-43], a reported clinical response rate for treatment of colorectal carcinoma with cetuximab (a chimeric monoclonal antibody targeting the extracellular domain of EGFR) is about 11% [New England J. Med. 2004; 351:337-45], and a reported clinical response rate for treatment of NSCLC with erlotinib is about 8.9% [13].

Thus, there is a need for predicting patient responsiveness to EGFR inhibitors prior to treatment with such agents, so as to better individualize patent therapy.

For example, Cetuximab (Erbitux® ImClone Systems) is a chimeric IgG1 monoclonal antibody that was developed to inhibit signaling through EGFR by competitively inhibiting the binding of EGF and other ligands of the receptor, thereby hindering phosphorylation of EGFR, and thus affecting several downstream signaling pathways involved in cancer [32]. However, phosphorylation status of EGFR or the downstream signaling molecules STAT3, Akt, and Erk has not been found to be predictive of a response to cetuximab [33].

Phase II and Phase III clinical trials of cetuximab monotherapy and in combination with chemotherapy for patients with metastatic colorectal or non-small cell carcinoma have identified a subset of patients for which cetuximab is of benefit; however, the patient responses did not always correlate with the tumor expression of EGFR as detected by immunohistochemistry (IHC) [34], which was used to qualify patients for these clinical trial [35, 36]. Additional work has revealed that some IHC EGFR-negative patients may benefit from cetuximab treatment as well [34, 37]. Excitement over the use this monoclonal antibody alone or in combination with chemotherapy has been tempered by its cost for treatment [38] and its modest but clinical benefit to select patients [39].

Thus, improved methods are needed to identify patients who may benefit from treatment with EGFR inhibitors, such as cetuximab [39].

### SUMMARY OF THE INVENTION

The present invention provides methods for individualizing chemotherapy for cancer treatment, and particularly for evaluating a patient's responsiveness to one or more epidermal growth factor receptor (EGFR) inhibitors prior to treatment with such agents. In one embodiment, the invention provides an in vitro chemoresponse assay for predicting a patient's response to a monoclonal antibody inhibitor of EGFR, such as cetuximab. The method generally comprises culturing malignant cells from a patient's specimen (e.g., biopsy specimen), contacting the cultured cells with an EGFR inhibitor that is a candidate treatment for the patient, and evaluating the cultured cells for a response to the drug. In certain embodiments, monolayer(s) of malignant cells are cultured from explants prepared by mincing tumor tissue, and the cells of the monolayer are suspended and plated for chemosenstivity testing. The in vitro response to the drug as determined by the method of the invention is correlative with the patient's in vivo response upon receiving the EGFR inhibitor during chemotherapeutic treatment (e.g., in the course of standardized or individualized chemotherapeutic regimen).

In certain embodiments, the EGFR inhibitor is a tyrosine kinase inhibitor such as erlotinib, gefitinib, or lapatinib. In other embodiments the EGFR inhibitor is a monoclonal antibody targeting EGFR, such as cetuximab. While such agents may have relatively low population response rates, the invention provides a convenient in vitro assay to predict whether a particular patient will be responsive to an EGFR inhibitor, thus avoiding ineffective treatment as well as unnecessary toxicity and expense. As described herein, the method of the invention predicts responsiveness to EGFR inhibitor, such as cetuximab, at a rate that matches reported clinical response rate for the EGFR inhibitor.

### DESCRIPTION OF THE FIGURES

FIG. 1 shows in vitro response of three human non-small cell lung cancer (NSCLC) cell lines (H292, Calu-3, H358) after a 72-hour treatment with erlotinib. H292 is Responsive to erlotinib, Calu-3 is Intermediate Responsive, and H358 is Non-Responsive.

FIG. 2 shows in vitro chemoresponse to erlotinib in primary cultures of lung cancer. In vitro response of 34 lung cancer tumors after a 72-hour treatment with erlotinib is shown. 3 (8.8%) specimens are Responsive to erlotinib, 7 (20.6%) are Intermediate Responsive, and 24 (70.6%) are Non-Responsive. These results are consistent with a reported clinical response rate of 8.9% in NSCLC patients [13].

FIG. 3 shows in vitro chemoresponse to: (A) four immortalized cell lines (SK-OV3, BT474, MDA-MB-231, MCF7), and (B) to 55 primary cultures of breast carcinomas. All four cell lines (BT474, MDA-MB-231, MCF7, SK-OV3) were responsive to lapatinib treatment, with EC50 values of approximately 10 uM. Dose-response curves of the 55 primary breast cultures revealed that 9% of the specimens tested were responsive to lapatinib, 15% had an intermediate response and 76% were non-responsive. These results are consistent with a reported clinical response rate of 10% for lapatinib in breast carcinoma [New England J. Med. 2006; 355:2733-43].

FIG. 4 shows in vitro chemoresponse to four different immortalized cell lines (NCI-H292, NCI-H522, NCI-H1666, Calu3), and to 54 primary cultures of human colorectal tumor specimens. Two of the examined cell lines showed response to cetuximab treatment; EC50 values for NCI-H292 and NCI-H1666 were 825 nM and 13 nM, respectively. NCI-14522 and Calu3 were non-responsive to cetuximab. Dose-response curves of the 54 primary colorectal cultures revealed that 8% of the cultures tested were responsive to cetuximab, 22% had an intermediate response, and 70% were non-responsive. These results are consistent with a reported clinical response rate of 11% for cetuximab in colorectal carcinoma patients [New England J. Med. 2004; 351:337-45].

FIG. 5 shows responses of human non-small cell lung carcinoma cell lines assayed by the ChemoFx® drug response marker. Cytotoxic index (CI) for each non-small-cell lung carcinoma (NSCLC) cell line over the range of doses from 0 to 1.6 μM listed as dose number. Each cell line was exposed to cetuximab for 72 h. The number of remaining cells living after cetuximab incubation was used to calculate a CI for each cell line at each dose. Adjusted area under the curve (aAUC) was calculated for each cell line and used to establish the categorical values: nonresponsive (NR), intermediate responsive (IR), and responsive (R).

FIG. 6 shows immunocytochemistry for EGFR expression in human immortalized non-small cell lung carcinoma cell lines intermediate responsive (HCC827) and non-responsive (H358) to cetuximab. A, Staining for EGFR (green fluorescence) and wheat germ agluttinin (WGA, red fluorescence) in HCC827 control and cetuximab-treated cells is shown. HCC827 cells have a mutant form of EGFR and are intermediate responsive to cetuximab. B, Staining for EGFR (green fluorescence) and wheat germ agluttinin (WGA, red fluorescence) in H358 control and cetuximab-treated cells is shown. H1358 cells contain a wild type EGFR. Original magnification, 10×.

FIG. 7 shows the expression of the external epitope of EGFR in cetuximab-treated cancer cell lines by: A, Western blot analysis of EGFR. β-actin expression was used as a loading control. B, Integrated intensity ratio of EGFR/β-actin from Western blot confirms the decrease in EGFR expression in cetuximab-treated cells. C. In-Cell Western analysis of the expression of the external epitope of EGFR, * indicates significance at <0.05.

FIG. 8 shows downstream signaling molecules in cancer cell lines exposed to cetuximab for 4 hours. ChemoFx cetuximab-intermediate responsive (HCC827) and nonresponsive (NCI-H520) cell lines were exposed to cetuximab for 4 hours and then assayed for total and phosphorylated protein expression of Erk (A) and Akt (B).

FIG. 9 shows in vitro chemoresponse of tumor specimens from patients with either colorectal or lung cancer. Primary cultures were established from specimens of colorectal cancer (A) and lung cancer (B). Specimens were tested for response to a 72-h cetuximab exposure using the ChemoFx drug response marker. The number of cells living after cetuximab treatment was used to calculate a cytotoxic index and the adjusted area under the curve (aAUC) was calculated for each primary culture to establish the categorical values: nonresponsive (NR), intermediate responsive (IR), and responsive (R).

FIG. 10 shows immunocytochemistry for EGFR expression in human immortalized non-small cell lung carcinoma cell lines non-responsive to cetuximab. Staining for EGFR (green fluorescence) and wheat germ agluttinin (WGA, red fluorescence) in H520 control and cetuximab-treated cells is shown.

### DETAILED DESCRIPTION OF THE INVENTION

The present invention provides methods for individualizing chemotherapy for cancer treatment, and particularly, provides an in vitro chemoresponse assay for evaluating a patient's responsiveness to one or more EGFR inhibitors prior to treatment with such agents. The method generally comprises culturing malignant cells from a patient's specimen (e.g., biopsy), contacting the cultured cells with an EGFR inhibitor, and evaluating the cultured cells for a response to the drug. The in vitro response to the drug as determined by the method of the invention is correlative with an in vivo response upon receiving the EGFR inhibitor during chemotherapeutic treatment.

### Chemoresponse Assay

The present invention supports individualized chemotherapy decisions for cancer patients, and particularly with candidate EGFR inhibitors. The patient generally has a cancer for which an EGFR is a candidate treatment, for example, alone or in combination with other therapy. For example, the cancer may be selected from breast, ovarian, colorectal, endometrial, thyroid, nasopharynx, prostate, head and neck, liver, kidney, pancreas, bladder, brain, and lung. In certain embodiments, the tumor is a solid tissue tumor and/or is epithelial in nature. For example, the patient may be a Her2-positive breast cancer patient, a colorectal carcinoma patient, NSCLC patient, head and neck cancer patient, or endometrial cancer patient.

The present invention involves conducting chemoresponse testing with one or a panel of chemotherapeutic agents on cultured cells from a cancer patient, including one or more EGFR inhibitors. In certain embodiments, the chemoresponse method is as described in U.S. Pat. Nos. 5,728,541, 6,900,027, 6,887,680, 6,933,129, 6,416,967, 7,112,415, and 7,314,731 (all of which are hereby incorporated by reference in their entireties). The chemoresponse method may further employ the variations described in U.S. Published Patent Application No. 2007/0059821 and U.S. Pat. No. 7,642,048, both of which are hereby incorporated by reference in their entireties. Such chemoresponse methods are commercially available as the ChemoFx® Assay (Precision Therapeutics, Inc, Pittsburgh, Pa.).

Briefly, in certain embodiments, cohesive multicellular particulates (explants) are prepared from a patient's tissue sample (e.g., a biopsy sample) using mechanical fragmentation. This mechanical fragmentation of the explant may take place in a medium substantially free of enzymes that are capable of digesting the explant. However, in some embodiments, some enzymatic treatment may be conducted. Generally, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm³, for example, about 1 mm³.

After the tissue sample has been minced, the particles are plated in culture flasks (e.g., about 5 to 25 explants per flask). For example, about 9 explants may be plated per T-25 flask, or about 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of a few days to a few weeks a cell monolayer will form. Further, it is believed (without any intention of being bound by the theory) that tumor cells grow out from the multicellular explant prior to stromal cells. Thus, by initially maintaining the tissue cells within the explant and removing the explant at a predetermined time (e.g., at about 10 to about 50 percent confluency, or at about 15 to about 25 percent confluency), growth of the tumor cells (as opposed to stromal cells) into a monolayer is facilitated. Further, in certain embodiments, the tumor explant may be agitated to substantially release tumor cells from the tumor explant, and the released cells cultured to produce a cell culture monolayer. The use of this procedure to form a cell culture monolayer helps maximize the growth of representative tumor cells from the tissue sample.

Prior to the chemotherapy assay, the growth of the cells may be monitored, and data from periodic counting may be used to determine growth rates which may or may not be considered parallel to growth rates of the same cells in vivo in the patient. If growth rate cycles can be documented, for example, then dosing of certain active agents may be customized for the patient. Monolayer growth rate and/or cellular morphology and/or epithelial character may be monitored using, for example, a phase-contrast inverted microscope. Generally, the monolayers are monitored to ensure that the cells are actively growing at the time the cells are suspended for drug exposure. Thus, the monolayers will be non-confluent when the cells are suspended for chemoresponse testing.

A panel of active agents may then be screened using the cultured cells, including one or more EGFR inhibitors, such as, for example, cetuximab. Generally, the agents are tested against the cultured cells using plates such as microtiter plates. For the chemosensitivity assay, a reproducible number of cells is delivered to a plurality of wells on one or more plates, preferably with an even distribution of cells throughout the wells. For example, cell suspensions are generally formed from the monolayer cells before substantial phenotypic drift of the tumor cell population occurs. The cell suspensions may be, without limitation, about 4,000 to 12,000 cells/ml, or may be about 4,000 to 9,000 cells/ml, or about 7,000 to 9,000 cells/ml. The individual wells for chemoresponse testing are inoculated with the cell suspension, with each well or “segregated site” containing about 10² to 10⁴ cells. The cells are generally cultured in the segregated sites for about 4 to about 30 hours prior to contact with an agent.

Each test well is then contacted with at least one pharmaceutical agent, or a sequence of agents. In addition to at least one EGFR inhibitor (as discussed in more detail below), the panel of chemotherapeutic agents may comprise at least one agent selected from a platinum-based drug, a taxane, a nitrogen mustard, a kinase inhibitor, a pyrimidine analog, a podophyllotoxin, an anthracycline, a monoclonal antibody, and a topoisomerase I inhibitor. For example, the panel may comprise 1, 2, 3, 4, or 5 agents selected from bevacizumab, capecitabine, carboplatin, cisplatin, cyclophosphamide, docetaxel, doxorubicin, epirubicin, etoposide, 5-fluorouracil, gemcitabine, irinotecan, oxaliplatin, paclitaxel, tamoxifen, topotecan, and trastuzumab, in addition to other potential agents for treatment. In certain embodiments, the chemoresponse testing includes one or more combination treatments, such combination treatments including one or more agents described above. Generally, each agent in the panel is tested in the chemoresponse assay at a plurality of concentrations representing a range of expected extracellular fluid concentrations upon therapy.

The efficacy of each agent in the panel is determined against the patient's cultured cells, by determining the viability of the cells (e.g., number of viable cells). For example, at predetermined intervals before, simultaneously with, or beginning immediately after, contact with each agent or combination, an automated cell imaging system may take images of the cells using one or more of visible light, UV light and fluorescent light. Alternatively, the cells may be imaged after about 25 to about 200 hours of contact with each treatment. The cells may be imaged once or multiple times, prior to or during contact with each treatment. Of course, any method for determining the viability of the cells may be used to assess the efficacy of each treatment in vitro.

While any grading system may be employed, in certain embodiments the grading system may employ from 2 to 10 response levels, e.g., about 3, 4, or 5 response levels. For example, when using three response grades, the three grades may correspond to a responsive grade, an intermediate responsive grade, and a non-responsive grade. In certain embodiments, the patient's cells show a heterogeneous response across the panel of agents, making the selection of an agent particularly crucial for the patient's treatment.

The output of the assay is a series of dose-response curves for tumor cell survivals under the pressure of a single or combination of drugs, with multiple dose settings each (e.g., ten dose settings). To better quantify the assay results, the invention employs in some embodiments a scoring algorithm accommodating a dose-response curve. Specifically, the chemoresponse data are applied to an algorithm to quantify the chemoresponse assay results by determining an adjusted area under curve (aAUC)

The aAUC takes into account changes in cytotoxicity between dose points along a dose-response curve, and assigns weights relative to the degree of changes in cytotoxicity between dose points. For example, changes in cytotoxicity between dose points along a dose-response curve may be quantified by a local slope, and the local slopes weighted along the dose-response curve to emphasize cytotoxic responses.

For example, aAUC may be calculated as follows.

Step 1: Calculate Cytotoxity Index (CI) for each dose, where CI=Mean_{drug}/Mean_{control}.

Step 2: Calculate local slope (S_{d}) at each dose point, for example, as S_{d}=(CI_{d}−CI_{d-1})/Unit of Dose, or S_{d}=(CI_{d-1}−CI_{d})/Unit of Dose.

Step 3: Calculate a slope weight at each dose point, e.g., W_{d}=1−S_{d}.

Step 4: Compute aAUC, where aAUC=ΣW_{d} CI_{d}, and where, d=each dose, e.g., 1, 2, . . . , 10. Equation 4 is the summary metric of a dose response curve and may used for subsequent regression over reference outcomes.

Usually, the dose-response curves vary dramatically around middle doses, not in lower or higher dose ranges. Thus, the algorithm in some embodiments need only determine the aAUC for a middle dose range, such as for example (where from 8 to 12 doses are experimentally determined, e.g., 10 doses), the middle 4, 5, 6, or 8 doses are used to calculate aAUC. In this manner, a truncated dose-response curve might be more informative in outcome prediction by eliminating background noise.

The numerical aAUC value (e.g., test value) may then be evaluated for its effect on the patient's cells, and compared to the same metric for other drugs on the patient's cells. For example, a plurality of drugs may be tested, and aAUC determined as above for each, to determine whether the patient's cells have a sensitive response, intermediate response, or resistant response to each drug. Further, the measures may be compared to determine the most effective drug.

In some embodiments, the agents are designated as, for example, sensitive, or resistant, or intermediate, by comparing the aAUC test value to one or more cut-off values for the particular drug (e.g., representing sensitive, resistant, and/or intermediate aAUC scores for that drug). The cut-off values for any particular drug may be set or determined in a variety of ways, for example, by determining the distribution of a clinical outcome within a range of corresponding aAUC reference scores. That is, a number of patient tumor specimens are tested for chemosenstivity/resistance to a particular drug prior to treatment, and aAUC quantified for each specimen. Then after clinical treatment with that drug, aAUC values that correspond to a clinical response (e.g., sensitive) and the absence of significant clinical response (e.g., resistant) are determined. Cut-off values may alternatively be determined from population response rates. For example, where a patient population is known to have a response rate of 30% for the tested drug, the cut-off values may be determined by assigning the top 30% of aAUC scores for that drug as sensitive. Further still, cut-off values may be determined by statistical measures.

### Protein Analysis

The response to EGFR inhibitor may be determined by evaluating the cells for protein expression, including the phosphorylation status of an EGFR signaling molecule. Protein expression may be evaluated alone or in combination with cytotoxic index as a measure of drug response. Methods for evaluating protein expression and/or phosphorylation include traditional Western Blotting and In-Cell Western analysis.

In-Cell Western analysis is an immunocytochemical assay which can be performed in a microplate. Like traditional Western Blot analysis, target-specific primary antibodies and labeled secondary antibodies are used to detect target proteins. However, in In-Cell Western analysis, target proteins are detected in fixed cells, thereby allowing detection of these signaling proteins in their cellular context.

In accordance with one aspect of the present invention, protein expression can be detected and/or analyzed by any method known in the art, for example immunocytochemistry, Western Blot analysis, or In-Cell Western analysis. In a particular embodiment, the external epitope of EGFR is detected in cultured cells by In-Cell Western analysis. In a further embodiment, downstream signaling molecules, for example, pSTAT3/STAT3, pERK/ERK and pAKT/AKT, are detected in cultured cells by In-Cell Western analysis.

### EGFR Inhibitors

In accordance with the present invention, cultured cells may be tested for their responsiveness to any candidate EGFR inhibitor (e.g., an EGFR inhibitor that is a candidate treatment for the patient). The EGRF inhibitor may be an EGFR tyrosine kinase inhibitor, or may alternatively target the extracellular domain of the EGFR target.

In certain embodiments, the EGFR inhibitor is a tyrosine kinase inhibitor such as Erlotinib, Gefitinib, or Lapatinib, or a molecule that targets the EGFR extracellular domain such as Cetuximab or Panitumumab.

Erlotinib hydrochloride (e.g., as marketed as Tarceva™) is used to treat non-small cell lung cancer, pancreatic cancer and several other types of cancer. Erlotinib specifically targets the epidermal growth factor receptor (EGFR) tyrosine kinase, which is highly expressed and occasionally mutated in various forms of cancer. It binds in a reversible fashion to the adenosine triphosphate (ATP) binding site of the receptor to inhibit receptor signaling. Erlotinib has shown a survival benefit in the treatment of lung cancer in phase III trials. It has been approved for the treatment of locally advanced or metastatic non-small cell lung cancer that has failed at least one prior chemotherapy regimen. The FDA has further approved the use of erlotinib in combination with gemcitabine for treatment of locally advanced, unresectable, or metastatic pancreatic cancer. It has been reported that responses among patients with lung cancer are seen most often in females who were never smokers, particularly Asian women and those with adenocarcinoma cell type.

Gefitinib acts in a similar manner to erlotinib (marketed as Tarceva), and is marketed under the trade name Iressa™. Research on gefitinib-sensitive non-small cell lung cancers has shown that a mutation in the EGFR tyrosine kinase domain may be responsible for activating anti-apoptotic pathways. These mutations may confer increased sensitivity to tyrosine kinase inhibitors such as gefitinib and erlotinib. Of the types of non-small cell lung cancer histologies, adenocarcinoma most often harbors these mutations. These mutations are more commonly seen in Asians, women, and non-smokers (who also tend to more often have adenocarcinoma).

Gefitinib is indicated for the treatment of locally advanced or metastatic non-small cell lung cancer (NSCLC) in patients who have previously received chemotherapy. There is also potential for use of gefitinib in the treatment of other cancers where EGFR overexpression is involved.

Lapatinib inhibits the tyrosine kinase activity associated with two oncogenes, EGFR (epidermal growth factor receptor) and HER2/neu (Human EGFR type 2). Over expression of HER2/neu can be responsible for certain types of high-risk breast cancers in women. Lapatinib is a protein kinase inhibitor shown to decrease tumor-causing breast cancer stem cells. Lapatanib inhibits receptor signal processes by binding to the ATP-binding pocket of the EGFR/HER2 protein kinase domain, preventing self-phosphorylation and subsequent activation of the signal mechanism.

Lapatinib is used as a treatment for women's breast cancer in patients who have HER2-positive advanced breast cancer that has progressed after previous treatment with other chemotherapeutic agents, such as anthracycline, taxane-derived drugs, or trastuzumab (Herceptin, Genentech).

Cetuximab is a chimeric monoclonal antibody targeting EGFR, and is given by intravenous injection for treatment of metastatic colorectal cancer and head and neck cancer. Cetuximab may act by binding to the extracellular domain of the EGFR, preventing ligand binding and activation of the receptor. This blocks the downstream signaling of EGFR resulting in impaired cell growth and proliferation. Cetuximab has also been shown to mediate antibody dependent cellular cytotoxicity (ADCC).

Cetuximab is used in metastatic colon cancer and is given concurrently with the chemotherapy drug irinotecan (Camptosar), a form of chemotherapy that blocks the effect of DNA topoisomerase I, resulting in fatal damage to the DNA of affected cells. Cetuximab was approved by the FDA for use in combination with radiation therapy for treating squamous cell carcinoma of the head and neck (SCCHN) or as a single agent in patients who have had prior platinum-based therapy.

Panitumumab is a recombinant, human IgG2 kappa monoclonal antibody that binds specifically to the human epidermal growth factor receptor (EGFR). Panitumumab is indicated as a single agent for the treatment of EGFR-expressing, metastatic colorectal carcinoma with disease progression on or following fluoropyrimidine-, oxaliplatin-, and irinotecan-containing chemotherapy regimens.

EGFR inhibitors are generally plagued by low population response rates, leading to ineffective or non-optimal chemotherapy in many instances, as well as unnecessary drug toxicity and expense. For example, a reported clinical response rate for treatment of breast carcinoma with lapatinib is 10% [New England J. Med. 2006; 355:2733-43], a reported clinical response rate for treatment of colorectal carcinoma with cetuximab is 11% [New England J. Med. 2004; 351:337-45], and a reported clinical response rate for treatment of NSCLC with erlotinib is 8.9% [13].

The method of the invention predicts patient responsiveness to EGFR inhibitors at rates that match reported clinical response rates for the EGFR inhibitors.

### EXAMPLES

### Example 1

### Erlotinib

Three human lung tumor-derived immortalized cell lines were tested in this study: H292, H358, and Calu3 (American Type Culture Collection, Manassas, Va.). These cell lines were seeded at 40,000 cells in T25 flasks (PGC Scientifics, Frederick, Md.) and allowed to grow for one week to approximately 90% confluence.

Patient tumor specimens: Primary cell cultures were established using tumor specimens procured for research purposes from the following sources: National Disease Research Interchange (Philadelphia, Pa.), Cooperative Human Tissue Network (Philadelphia, Pa.), Forbes Regional Hospital (Monroeville, Pa.), Jameson Hospital (New Castle, Pa.), Saint Barnabas Medical Center (Livingston, N.J.), Hamot Medical Center (Erie, Pa.), and Windber Research Institute (Windber, Pa.). The tumors were removed from the patient at the time of surgery, placed in the supplied 125-mL bottle containing sterile McCoy's shipping medium (Mediatech, Herndon, Va.), and shipped overnight to Precision Therapeutics, Inc. laboratories (Pittsburgh, Pa.).

Erlotinib hydrochloride was kindly provided by OSI Pharmaceuticals (Melville, N.Y.) as a lyophilized powder. The drug was reconstituted to 5 mM in 100% DMSO and frozen at −80° C.

Cell lines and tissue specimens were processed and tested with the ChemoFx® assay as described elsewhere [21]. Also see, U.S. Pat. Nos. 5,728,541, 6,900,027, 6,887,680, 6,933,129, 6,416,967, 7,112,415, and 7,314,731; and U.S. application Ser. Nos. 10/399,563, 11/504,098, 11/595,967, 11/713,662, and 11/785,984, all of which are hereby incorporated by reference in their entireties, and especially with regard to tissue processing and cell culturing techniques, and assays. Ten doses of erlotinib were prepared by serial dilution. The same 10-dose concentration range was used for the cell lines and the tissue specimens. For each dose, a cytotoxic index (CI) was calculated according to the following formula: CI=Mean cell count_{dose x}/mean cell count_{control}, which represents the ratio of cells killed as a result of the treatment. Cell counts were the average of 3 replicates at each dose for primary cultures and 9 replicates at each dose for immortalized cell lines. Dose response curves were generated using the CI at each dose. Adjusted areas under the curve (aAUC) were calculated for each dose-response curve as previously described [20]. Assay results were classified as responsive (R; assay score ≦5.78), intermediate responsive (IR; assay score 5.79-6.98), or non-responsive (NR; assay score ≧6.99).

### Results

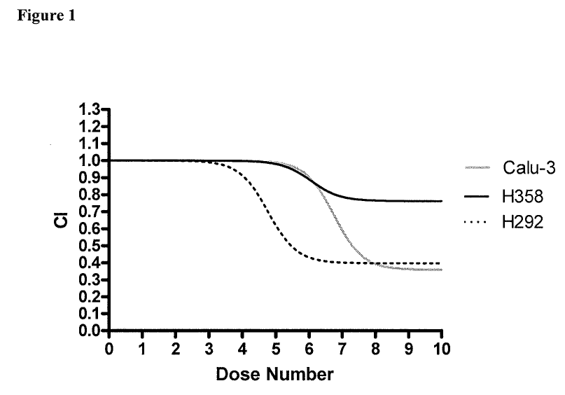

The chemoresponse assay was performed on 3 NSCLC lung cancer cell lines (H292, 11358, and Calu-3) to determine whether the assay was able to detect sensitivity of the cells to erlotinib. The 3 cell lines exhibited a heterogeneous response to erlotinib (FIG. 1). The assay prediction of response for H358 was non-responsive (NR), for Calu-3 was intermediate responsive (IR), and for H292 responsive (R).

TABLE I
Comparison of response to erlotinib treatment:
in vitro chemoresponse assay and ex vivo human
tumor xenograft outcomes on NSCLC cell lines.

| Cell | ChemoFx Assay | Xenograft TGI (%) |
|---|---|---|
| Line | Designation | [22] |
| H292 | R | 85 |
| Calu3 | IR | 67 |
| H358 | NR | 25 |

R = responsive, IR = intermediate responsive, NR = non-responsive; TGI = tumor growth inhibition

Each dose-response curve includes 9 replicates at each dose for each cell line tested; each assay included 3 replicates, and 3 assays were run per cell line. The coefficient of variance (CoV) was calculated for each cell line using the Log EC50 values (by dose number) for each assay. H292 had a CoV of 7%, H358 was 9%, and Calu-3 was 3%.

TABLE II
Coefficient of Variance for the ChemoFx Assay in evaluating
response to Erlotinib in 3 NSCLC cell lines.

| | Mean | |
|---|---|---|
| Cell Line | (Log EC50*) | CoV |
| H292 | 4.731 | 7% |
| Calu3 | 6.715 | 3% |
| H358 | 5.925 | 9% |

*By dose number
CoV = coefficient of variance

The in vitro responsiveness to erlotinib of the 3 NSCLC cell lines was compared with published reports of the responsiveness of human tumor xenografts [22]. The sensitivity of tumor growth inhibition in xenografts derived from these cell lines is consistent with the in vitro prediction of response in the same cell lines (Table I).

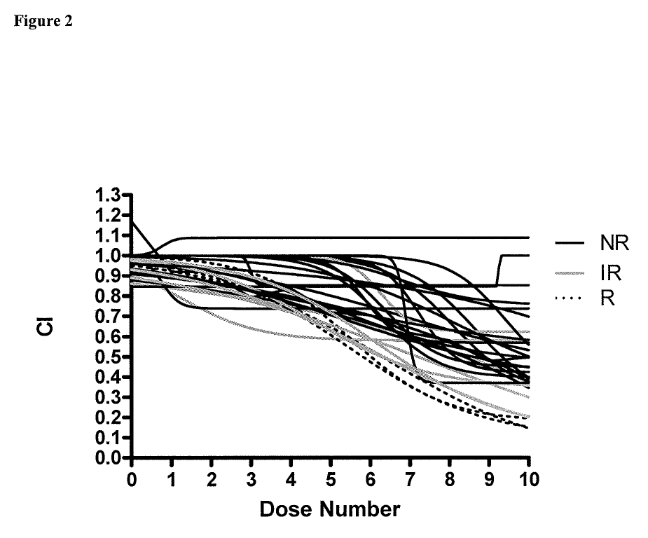

Of the 34 lung cancer patient specimens evaluated in this study, 22 (64.7%) were confirmed to be NSCLC, 11 (32.4%) were of unconfirmed lung cancer subtype, and 1 (2.9%) was confirmed as not NSCLC (mesothelioma). The 34 tumor specimens exhibited heterogeneity of in vitro response to erlotinib (FIG. 2). Of the 34 patient specimens, 3 (8.8%) were assay responsive to erlotinib, 7 (20.6%) were intermediate responsive, and 24 (70.6%) were non-responsive.

These results indicate that the assay described herein is able to distinguish tumor response to erlotinib in patients with lung carcinoma. The invention is thus useful as a decision support tool to assist oncologists in making treatment decisions involving erlotinib in lung cancer patients. The approach described above was to first conduct the assay on NSCLC cell lines to determine its ability to distinguish in vitro response to erlotinib. The responses of the three NSCLC cell lines tested (H292, H358, Calu-3) to erlotinib in the current study were similar to the responses observed in previously published studies using other types of chemoresponse assays [23,24]. In addition, the assay is shown to be highly reproducible (i.e. low process variability) in assessing chemoresponse to erlotinib in 3 separate NSCLC cell lines.

To confirm the range of responses observed, we next compared the in vitro sensitivity of these cell lines to the observed outcomes of ex vivo human tumor xenografts derived from those same cell lines as an estimation of correlation with clinical response. Corresponding sensitivities support the hypothesis that in vitro response may predict clinical response.

Having evidence that the assay prediction of response to erlotinib of the NSCLC cell lines corresponded to responsiveness of human tumor xenografts produced from the same cell lines, we next examined human lung tumor specimens. The assay was able to distinguish sensitivity to erlotinib among 34 human tumor specimens. Our finding that 8.8% of the tumors were responsive to erlotinib is similar to the 8.9% reported response rate in a phase 3, randomized, double blind, placebo-controlled study of previously treated NSCLC patients [13].

Currently, erlotinib is FDA approved only as second- or third-line treatment for advanced NSCLC. Reports from clinical trials to date have not shown a benefit of erlotinib in first line treatment [8-11]. However, subgroup analyses have shown that groups of patients differed in their sensitivity and clinical response to erlotinib [10,13,25]. Investigators have speculated about the potential findings had the large, first-line studies been conducted on selected populations showing increased sensitivity [6]. Thus, an accurate and reliable test to identify erlotinib-sensitive subpopulations of NSCLC patients would be of crucial benefit. Much interest has been focused on identifying patients sensitive to EGFR inhibition using molecular profiles, such as EGFR mutations and amplifications as well as increased gene number [26,27]. A chemoresponse assay that can reproducibly and reliably identify sensitive patients by in vitro tumor response would be a superior alternative to support clinical decision making, in some embodiments, may be performed alongside molecular profiling.

### Example 2

### Lapatinib

Lapatinib (Tykerb®) is a small molecule tyrosine kinase inhibitor which targets the intracellular domain of both the epidermal growth factor receptor and Her2, thereby inhibiting cell growth and proliferation. Lapatinib is currently FDA-approved to treat Her2 positive breast cancer which has previously been treated with anthracycline and taxane therapies and trastuzumab. Due to the low population response rate of lapatinib, a biomarker which can identify patients with an increased likelihood for response would be of great clinical utility. This example demonstrates an in vitro chemoresponse assay developed to predict sensitivity and resistance of primary cultures of human breast tumor specimens to lapatinib.

The chemoresponse assay for lapatinib was developed using four different immortalized cell lines (SK-OV3, BT474, MDA-MB-231, MCF7). In addition to cell lines, the chemoresponse assay was also performed on 55 primary cultures of breast carcinomas. All cultures were confirmed to contain keratin-positive epithelial cells using fluorescence immunocytochemistry. Cell lines and specimens were treated with a 10 dose concentration range of lapatinib for 72 hours and stained with DAPI; remaining live cells were counted on an inverted fluorescent microscope. Resulting dose-response curves were analyzed and categorized as responsive, intermediate responsive, or non-responsive.

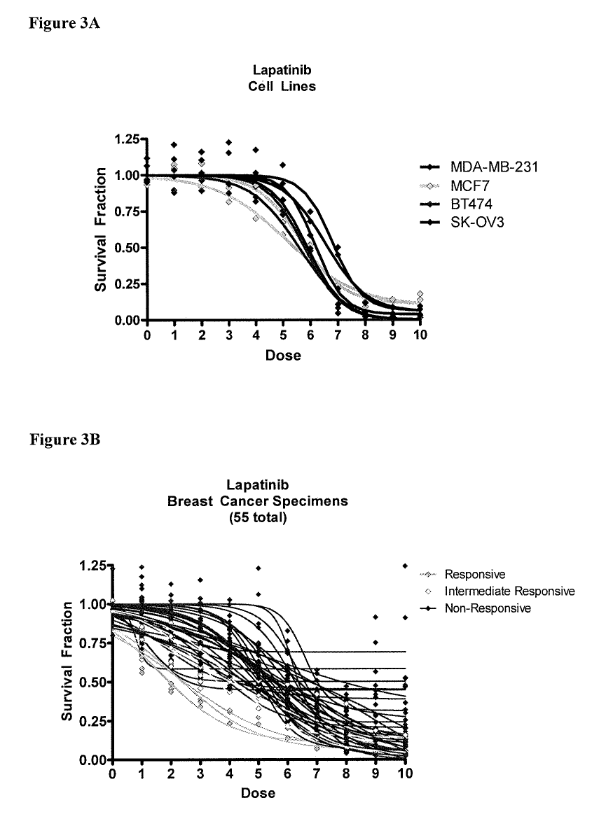

All four cell lines (BT474, MDA-MB-231, MCF7, SK-OV3) were responsive to lapatinib treatment, with EC50 values of approximately 10 uM (FIG. 3A). Dose-response curves of the 55 primary breast cultures revealed that 9% of the specimens tested were responsive to lapatinib, 15% had an intermediate response and 76% were non-responsive. These results are consistent with a reported clinical response rate of 10% (FIG. 3B).

In conclusion, these results demonstrate that in vitro chemoresponse testing is useful in predicting patient response to lapatinib. This test may increase the efficacy of the current chemotherapy decision-making process for patients.

### Example 3

### Cetuximab

Cetuximab (Erbitux®) is a chimeric monoclonal antibody that binds to the extracellular domain of the epidermal growth factor receptor. This interaction interferes with binding of the ligand and causes internalization of the receptor which blocks the downstream signaling of EGFR, resulting in impaired cell growth and proliferation. Cetuximab has also been shown to mediate antibody dependent cellular cytotoxicity. Cetuximab is FDA-approved to treat head and neck cancer and colorectal carcinomas; it is also being evaluated in clinical trials for use in other cancers, including non-small cell lung and endometrial cancer. Due to the low population response rate of cetuximab, a test that can identify patients with an increased likelihood for response would be of great clinical utility. The current example demonstrates an in vitro chemoresponse assay to predict response of primary cultures of human colorectal tumor specimens to cetuximab.

### Methods and Results

The chemoresponse assay was developed using four different immortalized cell lines (NCI-H292, NCI-H522, NCI-H1666, Calu3). The chemoresponse assay was also performed on 54 primary cultures of human colorectal tumor specimens. Cell lines and specimens were treated with a 10 dose concentration range, between 6.3 pM-1.64 μM, of cetuximab for 72 hours, stained with a nuclear dye, and remaining post-treatment live cells were counted. Resulting dose-response curves were analyzed.

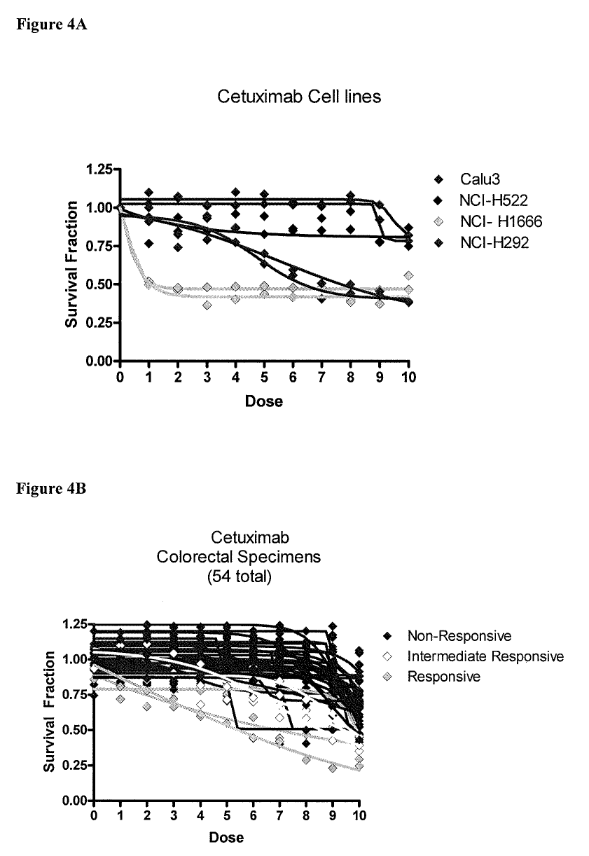

Two of the examined cell lines showed response to cetuximab treatment; EC50 values for NCI-H292 and NCI-H1666 were 825 nM and 13 nM, respectively (FIG. 4A). NCI-H522 and Calu3 were deemed non-responsive to cetuximab. Dose-response curves of the 54 primary colorectal cultures revealed that 8% of the cultures tested were responsive to cetuximab, 22% had an intermediate response, and 70% were deemed non-responsive (FIG. 4B). These results are consistent with a reported clinical response rate of 11% for cetuximab in colorectal carcinoma patients.

In a second experiment, four human lung tumor-derived immortalized cell lines were identified based on EGFR status (Table III). These included H520, H358, H1666 and HCC827 (American Type Culture Collection, Manassas, Va.). Each cell line was seeded at 40,000 cells in a T25 flask (Greiner Bio-One International AG, Kremsmünster, Austria) in Roswell Park Memorial Institute 1640 (RPMI 1640) medium (Mediatech, Manassas, Va.) containing 10% fetal bovine serum (FBS) (Hyclone, Logan, Utah, USA) and grown for 1 week to approximately 90% confluence.

Patient tumor specimens. Deidentified colorectal and lung cancer specimens were procured for research purposes from the National Disease Research Interchange (Philadelphia, Pa.), Cooperative Human Tissue Network (Philadelphia, Pa.), Forbes Regional Hospital (Monroeville, Pa.), Jameson Hospital (New Castle, Pa.), Saint Barnabas Medical Center (Livingston, N.J.), Hamot Medical Center (Erie, Pa.) and Windber Research Institute (Windber, Pa.). Tumor type was provided as either colorectal or NSCLC or other lung cancer. Small-cell lung carcinomas or mesothelioma were not included. Stage for any specimen was not available.

After surgical removal, each specimen was placed in a 125-mL bottle containing sterile McCoy's shipping medium (Mediatech, Manassas, Va.) and shipped overnight to the research laboratory at Precision Therapeutics, Inc (Pittsburgh, Pa.).

TABLE III
Comparison of EGFR status in each cell line used for
ChemoFx determination of cetuximab sensitivity.

| Cell Line (ATCC Name) | EGFR Status | Source |
|---|---|---|
| NCI-H358 (CRL 5807) | Wild-type | [41] |
| NCI-H1666 (CRL 5885) | Wild-type | [43] |
| NCI-H520 (HTB 182) | Null | [41] |
| HCC827 (CRL 2868) | Mutant | [43] |

Cell lines and tissue specimens were processed and tested with the ChemoFx® assay as described elsewhere [21]. Also see, U.S. Pat. Nos. 5,728,541, 6,900,027, 6,887,680, 6,933,129, 6,416,967, 7,112,415, and 7,314,731; and U.S. application Ser. Nos. 10/399,563, 11/504,098, 11/595,967, 11/713,662, and 11/785,984, all of which are hereby incorporated by reference in their entireties, and especially with regard to tissue processing and cell culturing techniques, and assays. Ten doses of cetuximab were prepared by serial dilution in RPMI-1640 with 10% FBS and ranged from 6.3 pM to 1.6 μM. The same 10-dose concentration range was used for the cell lines and the tissue specimens. For each dose, a cytotoxic index (CI) was calculated according to the following formula: CI=Mean cell count_{dose x}/mean cell count_{control}, which represents the ratio of cells killed as a result of the treatment. Cell counts were the average of 3 replicates at each dose for primary cultures and 9 replicates at each dose for immortalized cell lines. Dose response curves were generated using the CI at each of 5 doses contained within the 10 dose range. Adjusted areas under the curve (aAUC) were calculated for each dose-response curve as previously described [20] and used to set cutoff values for the categorical responses. Assay results were classified as responsive (R; aAUC<5.78), intermediate responsive (IR; aAUC 5.79-6.98), or non-responsive (NR; aAUC>6.99).

Immunohistochemistry Each cell line was plated at 800 cells/well in 384-well microtiter plates and incubated overnight at 37° C. in 5% CO₂. Each cell line was cultured in RPMI 1640 containing 10% FBS as well as BEGM containing 5% FBS. Each cell line in each medium type was treated with cetuximab (1.6 μM) or vehicle for 24 hours. Cells were fixed in 4% paraformaldehyde (Sigma-Aldrich, St. Louis, Mo.), washed 4 times with phosphate buffered saline (PBS) (LI-COR Biotechnology, Lincoln, Nebr.) at room temperature with gentle agitation for 5 minutes and incubated at room temperature with Odyssey blocking buffer (LI-COR Biosciences) with gentle agitation for 1 hour to reduce the nonspecific antibody binding. Then, the cells were incubated overnight at 4° C. with the mouse monoclonal antibody to EGFR (1:200; NeoMarkers, Fremont, Calif.) with gentle agitation. The cells were washed with PBS and incubated at room temperature for 1 hour with wheat germ agglutinin (WGA) (Invitrogen, Carlsbad, Calif.). The cells were washed with PBS and incubated at room temperature for 45 minutes with Alexa Fluor 488-conjugated secondary antibody (Invitrogen, Carlsbad, Calif.). Images were acquired by using a Nikon Eclipse TE300 inverted microscope (Automated Cell Inc., Pittsburgh, Pa.), equipped with a ProgRes C5 cool camera (Jenoptik Inc., Easthampton, Mass.) and analyzed for staining intensity.

Western Blotting. To determine the specificity of the anti-EGFR antibody, the HCC827 and H520 cell lines were plated at 2.5×10⁵ cells in T25 flasks and cultured at 37° C. in 5% CO2 in RPMI 1640 containing 10% FBS and 1% penicillin streptomycin (Mediatech) for 3 days before drug treatment. Each cell line was treated for 24 hours with an intermediate (1.6 nM) and a high (1.6 μM) cetuximab concentration as identified by the results of the ChemoFx assay and then assessed for total external EGFR protein expression by Western blotting. Control flasks were treated with RPMI complete medium containing no drug. Cells were washed twice with cold PBS, scraped from the flask with 2× Lamelli Buffer (Bio-Rad laboratories, Hercules, Calif.) with 5% β-mercaptoethanol (Sigma-Aldrich, St. Louis, Mo.) and 0.05% protease inhibitor cocktail (Sigma-Aldrich). An RC DC protein assay (Bio-Rad, Hercules, Calif.) was performed according to manufacturer's instructions to determine the total protein level present in each sample. Protein samples were loaded at 50 μg/lane, subjected to SDS-PAGE and transferred to nitrocellulose membranes (Pall Corporation, Pensacola, Fla.). Membranes were incubated overnight with mouse primary monoclonal antibody to the external epitope of EGFR (1:200; NeoMarkers). Membranes were then washed with 0.1% Tween-20 in PBS (PBST, Fisher Scientific, Pittsburgh, Pa.) and incubated with the secondary antibody IRDye 800CW-labeled goat anti-mouse (1:800; LI-COR). Membranes were imaged on a LI-COR Odyssey Infrared Scanning System and analyzed using the Odyssey software (v. 3.0, LI-COR). The membranes were striped and reprobe for β-actin so that signals were normalized to β-actin (Cell Signaling Technology, Beverly, Mass.).

In-Cell Western assays. HCC827, H520, H1666 and H358 cell lines were plated in a 384-well microtiter plate at 320 cells/well and cultured for 3 days at 37° C. in 5% CO2 before drug treatment. Each cell line was cultured in RPMI 1640 containing 10% FBS. After 3 days in culture, each cell line was treated with cetuximab (1.6 μM and 1.6 nM) for 2, 4 and 24 hours. After treatment, cells were fixed with 4% paraformaldehyde. Cells (except for those used to detect the external EGFR staining) were permeabilized with 0.1% Triton X-100 (Fisher Scientific) in PBS. All cells were then washed twice with PBS at room temperature and incubated for 1 h at room temperature in Odyssey blocking buffer with gentle agitation to reduce the nonspecific antibody binding. Then, the cells were incubated overnight at 4° C. with primary antibody diluted in Odyssey blocking buffer with gentle agitation. The primary antibodies were mouse monoclonal antibody to the external epitope of EGFR (1:200); rabbit anti-phospho Akt (1:200), mouse anti-Akt (1:100), rabbit anti-phospho Erk (1:200), mouse anti-Erk (1:50), rabbit anti-phospho STAT3 (1:400) and mouse anti-STAT3 (1:50) (all from Cell Signaling Technology); and mouse anti-phospho EGFR (1:50) and goat anti-EGFR (1:50) (Santa Cruz Biotechnology, Santa Cruz, Calif. USA). After incubation with primary antibody, the cells were washed 4 times with PBS and incubated for 1 hour with the appropriate IR-labeled secondary antibodies [IRDye 800CW-labeled goat anti-rabbit (1:800), IRDye 800CW-labeled goat anti-mouse (1:800), IRDye 800CW-labeled donkey anti-mouse (1:800), IRDye 680-labeled goat anti-mouse (1:400), IRDye 680-labeled donkey anti-goat (1:400), LI-COR] in 0.1% Tween-20 in Odyssey blocking buffer. Cells were then washed 4 times using PBST for 5 minutes with gentle agitation. After the last PBST wash, all liquid was removed from the wells and the plates were immediately scanned on a LI-COR Odyssey Infrared Imaging System and analyzed using Odyssey software and Microsoft Excel (v. 2003, Microsoft Corporation, Redmond, Wash., USA). Integrated intensity ratios were calculated for each cell line by subtracting the background intensity from the negative control intensity for the channel and then dividing the intensity at the 800 channel (green) by the intensity at the 700 channel (red).

### Results

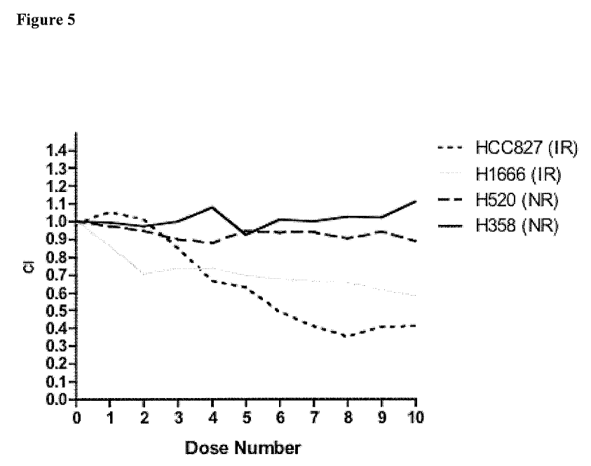

The chemoresponse assays was preformed on the 4 NSCLC lung cancer cell lines to determine whether the assay was able to detect sensitivity of the cells to cetuximab. Both the NCI H520 (EGFR null) and NCI H358 (EGFR wild type) cell lines were found to be non-responsive to cetuximab in the ChemoFx DRM, with aAUC values of 9.16 and 10.29, respectively. While the NCI-H1666 (EGFR wild type) and HCC827 (EGFR mutant) cell lines were found to be intermediate responsive (aAUC values of 6.62 and 5.92, respectively) based on the dose-dependent reduction in their cytotoxic index (FIG. 5).

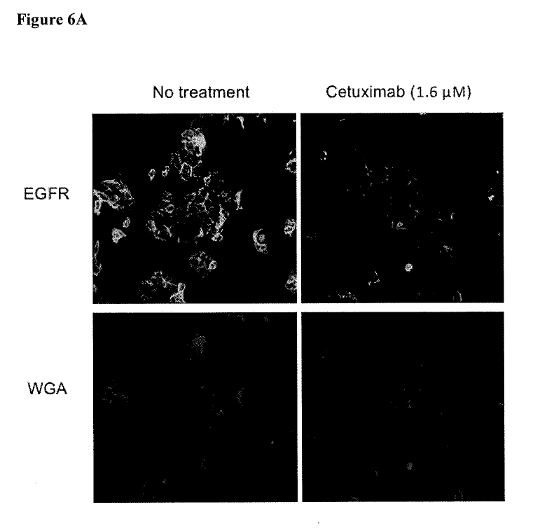

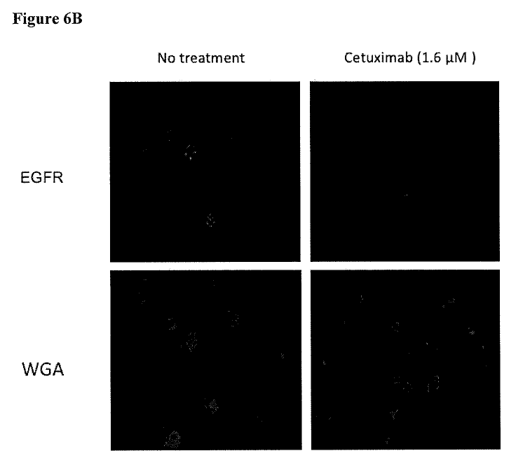

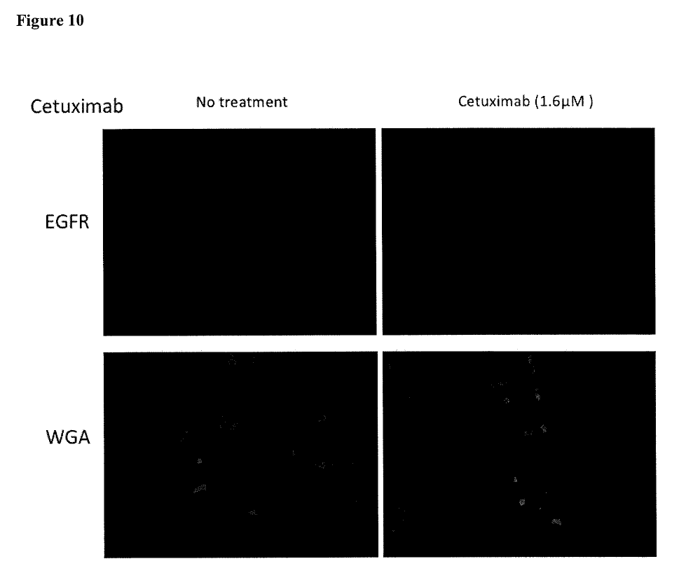

Immunocytochemistry for EGFR expression revealed that the ChemoFx-determined cetuximab-IR HCC827 cells had strong innate EGFR expression and that treatment with 1.6 μM cetuximab for 24 hours inhibited that expression (FIG. 6A). In contrast, ChemoFx cetuximab-NR H358 cell line had no change in EGFR expression with treatment of cetuximab for 24 hours (FIG. 6B). Similar results were also found when EGFR expression was examined in the other ChemoFx cetuximab-NR cell line (FIG. 10).

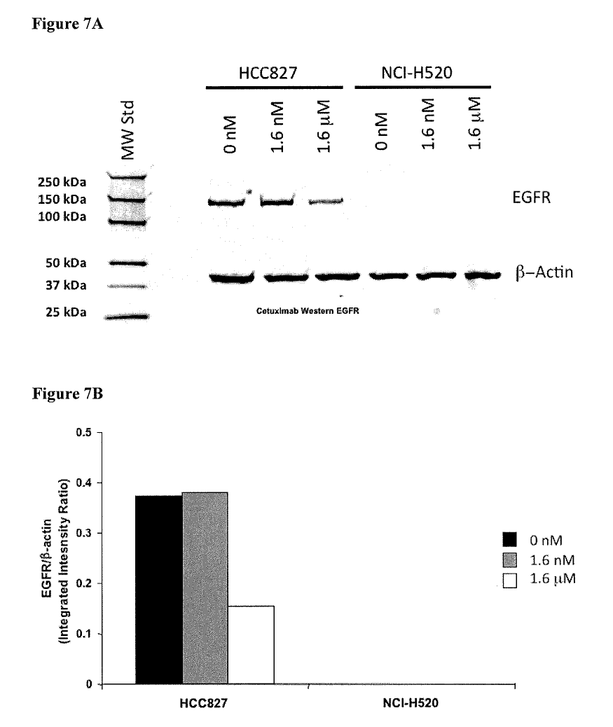

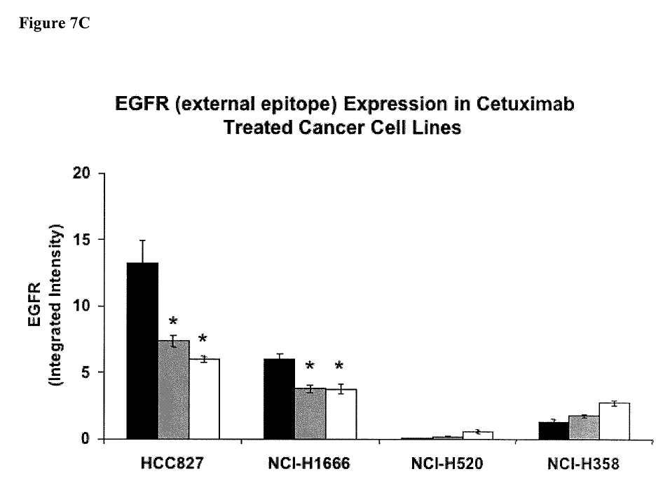

The specificity of the anti-EGFR antibody was examined using one ChemoFx cetuximab-IR cell line (HCC827) and one cetuximab-NR cell line (H520). Western blot analysis of EGFR expression after exposure to 0, 1.6 nM or 1.6 μM cetuximab for 24 hours revealed a cetuximab dose-dependent decrease in EGFR expression in HCC827 cells, while no EGFR expression was detected in H520 cells (FIG. 7) indicating that the anti-EGFR antibody was specific to detect the external epitope of the EGFR. Thus, we used this antibody to determine the effect of cetuximab on expression of several downstream signaling molecules using In-Cell Western analyses.

Results from the In-Cell Western analysis of EGFR expression were similar to the results from Western blotting for HCC827 and H520 (FIG. 7C). In addition, we examined H1666 and 1-1358 cell lines for EGFR expression and found that H1666 cells, similar to HCC827 cells, demonstrated a significant reduction in EGFR expression at both cetuximab concentrations (P<0.05), whereas cetuximab treatment in the two ChemoFx-NR cell lines did not affect EGFR expression.

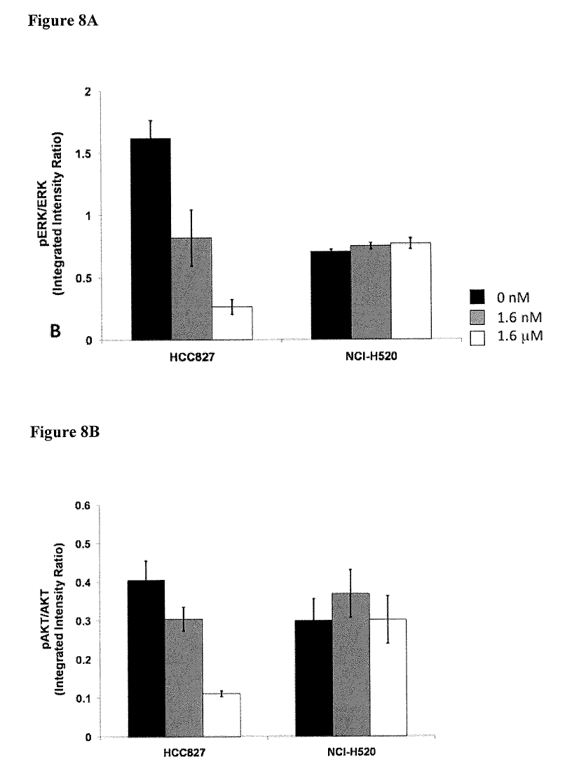

Examination of downstream signaling revealed that both HCC827 cells and H520 cells had no changes in the normalized expression of pEGFR and pSTAT3 at any time point. However, for the ChemoFx cetuximab-IR HCC827 cell line, the normalized expression of both pErk and pAkt was lower in cells treated with either 1.6 nM or 1.6 μM cetuximab for 4 h than in control cells (FIGS. 8A and B). Similar results were found for pErk expression at 2 and 24 h after cetuximab treatment; however, no change in pAkt expression was found at 2 or 24 h after treatment. For the ChemoFx cetuximab-NRH 520 cell line no changes in the normalized expression of either pErk or pAkt were detected in response to cetuximab treatment at any time point (FIGS. 8A and B).

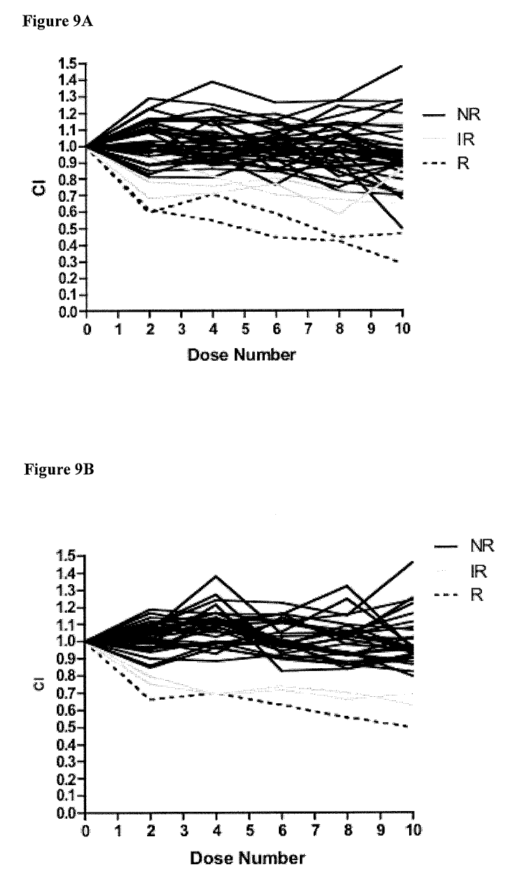

After a 72 hour exposure to various doses of cetuximab, the primary colorectal and lung cancer cultures were assayed using the ChemoFx DRM [21]. Of the 67 colorectal carcinoma specimens received and tested using ChemoFx, 2 primary cultures (3%) were R, 3 (4.5%) were IR, and 62 (92.5%) were NR to cetuximab (FIG. 9A). Of the 34 specimens from lung cancer patients, 1 primary lung culture (3%) was R, 2 (6%) were IR, and 31 (91%) were NR (FIG. 9B).

### CONCLUSIONS

The ChemoFx DRM was highly reproducible and was able to differentiate between cetuximab-IR and -NR cell lines. These ChemoFx data were confirmed with immunocytochemical and Western blot analyses for EGFR expression and the expression of EGFR to treatment with cetuximab. Data from In-Cell Western assays also support the effects of cetuximab on EGFR expression in cetuximab-IR cell lines. Furthermore, the ChemoFx DRM was able to identify patient tumor samples that were responsive to cetuximab.

Similar to the data from other studies [33,34], our data from NSCLC cell lines indicate that cetuximab sensitivity as determined by the ChemoFx DRM does not correlate with EGFR expression. The H358 cell line was NR to cetuximab even though the cells contain a wild-type EGFR. In contrast, both the H1666 cell line, which has a wild-type EGFR, and the HCC827 cell line, which has a mutant form of EGFR, were both sensitive to cetuximab, indicating that EGFR expression level as determined by In-Cell Western analysis and EFGR mutation status did not correlate with ChemoFx-determined sensitivity to cetuximab in these NSCLC cell lines. This lack of correlation between EGFR expression level and cetuximab sensitivity was reported for four other NSCLC cell lines [42]. Two reports from clinical trials have also identified a lack of correlation between IHC-determined EGFR status and cetuximab sensitivity in a subset of study participants [34,37]. Our data suggest that ChemoFx DRM may be useful to identify those tumors that may be sensitive to cetuximab regardless of EGFR status.

While treatment with cetuximab did not affect the expression of pEGFR and pSTAT3 in either ChemoFX cetuximab-IR or NR cell line tested, treatment did decrease expression of both pErk and pAkt in the ChemoFx cetuximab-IR cell line HCC827, but had no affect on the H520 cell line, which was found NR to cetuximab in the ChemoFx DRM. A similar lack of cetuximab effect on pEGFR has been reported for HCC827 cells, although cetuximab treatment did inhibit growth of these cells [43]. Other studies have also identified cell line-specific responses of pErk and pAkt and sensitivity to cetuximab treatment [33,42], supporting the concept that cetuximab induces inhibition of signaling molecules involved in cell proliferation in cetuximab-sensitive cell lines.

The ChemoFx cetuximab-sensitivity rate (combination of R and IR data) for samples from patients with NSCLC (9%) was similar to the published response rates for treatment of patients with NSCLC. A preliminary report of a Phase II trial testing the efficacy of cetuximab monotherapy indicated that 6% (2 out of 29) of the patients had a partial response [44], but final results have yet to be reported. A similar overall response rate was reported for an open-label phase II study to determine the efficacy of single agent cetuximab in patients with recurrent or progressive NSCLC; the overall response rate was 4.5% [45]. Patient response rates tended to be higher when cetuximab is combined with chemotherapy treatments for NSCLC [reviewed by (46)].

In addition, the ChemoFx cetuximab-sensitivity rate for samples from patients with colorectal cancer (7.5%) was similar to published response rates for treatment of colorectal cancer. A Phase IIB study comparing cetuximab monotherapy to cetuximab plus irinotecan treatment in irinotecan-refractory patients found that 10.8% (12 out of 111) of the patients had a partial response to cetuximab monotherapy whereas combination with irinotecan produced a partial response in 22% of the patients [35]. Saltz et al. reported similar a partial response rate (9%) in the open-label Phase II study to evaluate antitumor activity and toxicity of cetuximab monotherapy in chemotherapy refractory patients [36]. Similar to the patient response rates for treatment of NSCLC, response rates for treatment of colorectal cancer were higher when combined with various chemotherapy treatments [reviewed by (47)].

### REFERENCES

The following references are hereby incorporated by reference in their entireties for all purposes:
1. American Cancer Society Cancer Facts & Figures 2008. 2008;
2. Jemal A, Siegel R, Ward E et al. Cancer statistics, 2008. CA Cancer J Clin 2008; 58:71-96.
3. Schiller J H, Harrington D, Belani C P et al. Comparison of four chemotherapy regimens for advanced non-small-cell lung cancer. N Engl J Med 2002; 346:92-98.
4. Sandler A, Gray R, Perry M C et al. Paclitaxel-carboplatin alone or with bevacizumab for non-small-cell lung cancer. N Engl J Med 2006; 355:2542-2550.
5. Giaccone G HER1/EGFR-targeted agents: predicting the future for patients with unpredictable outcomes to therapy. Ann Oncol 2005; 16:538-548.
6. Sequist L V, Lynch T J EGFR Tyrosine Kinase Inhibitors in Lung Cancer: An Evolving Story. Annu Rev Med 2008; 59:429-442.
7. Ciardiello F, Tortora G EGFR antagonists in cancer treatment. N Engl J Med 2008; 358:1160-1174.
8. Giaccone G, Herbst R S, Manegold C et al. Gefitinib in combination with gemcitabine and cisplatin in advanced non-small-cell lung cancer: a phase III trial—INTACT 1. J Clin Oncol 2004; 22:777-784.
9. Herbst R S, Giaccone G, Schiller J H et al. Gefitinib in combination with paclitaxel and carboplatin in advanced non-small-cell lung cancer: a phase III trial-INTACT 2. J Clin Oncol 2004; 22:785-794.
10. Herbst R S, Prager D, Hermann R et al. TRIBUTE: a phase III trial of erlotinib hydrochloride (OSI-774) combined with carboplatin and paclitaxel chemotherapy in advanced non-small-cell lung cancer. J Clin Oncol 2005; 23:5892-5899.
11. Gatzemeier U, Pluzanska A, Szczesna A et al. Phase III study of erlotinib in combination with cisplatin and gemcitabine in advanced non-small-cell lung cancer: the Tarceva Lung Cancer Investigation Trial. J Clin Oncol 2007; 25:1545-1552.
12. Thatcher N, Chang A, Parikh P et al. Gefitinib plus best supportive care in previously treated patients with refractory advanced non-small-cell lung cancer: results from a randomised, placebo-controlled, multicentre study (Iressa Survival Evaluation in Lung Cancer). Lancet 2005; 366:1527-1537.
13. Shepherd F A, Rodrigues P J, Ciuleanu T et al. Erlotinib in previously treated non-small-cell lung cancer. N Engl J Med 2005; 353:123-132.
14. Eberhard D A, Giaccone G, Johnson B E Biomarkers of response to epidermal growth factor receptor inhibitors in Non-Small-Cell Lung Cancer Working Group: standardization for use in the clinical trial setting. J Clin Oncol 2008; 26:983-994.
15. Paez J G, Janne P A, Lee J C et al. EGFR mutations in lung cancer: correlation with clinical response to gefitinib therapy. Science 2004; 304:1497-1500.
16. Pao W, Miller V, Zakowski M et al. EGF receptor gene mutations are common in lung cancers from “never smokers” and are associated with sensitivity of tumors to gefitinib and erlotinib. Proc Natl Acad Sci USA 2004;101:13306-13311.
17. Sequist L V, Joshi V A, Janne P A et al. Response to treatment and survival of patients with non-small cell lung cancer undergoing somatic EGFR mutation testing. Oncologist 2007; 12:90-98.
18. Gallion H, Christopherson W A, Coleman R L et al. Progression-free interval in ovarian cancer and predictive value of an ex vivo chemoresponse assay. Int J Gynecol Cancer 2006; 16:194-201.
19. Herzog T J, Fader A N, Fensterer J E et al. A chemoresponse assay and survival in primary ovarian cancer. J Clin Oncol 2008; ASCO Annual Meetings Proceedings:
20. Mi Z, Holmes F A, Hellerstedt B et al. Feasibility assessment of a chemoresponse assay to predict pathologic response in neoadjuvant breast cancer. Anticancer Res 2008; 28:1733-40.
21. Brower S L, Fensterer J E, Bush J E The ChemoFx® assay: an ex vivo chemosensitivity and resistance assay for predicting patient response to cancer chemotherapy. 2008;57-78.
22. Thomson S, Buck E, Petti F et al. Epithelial to mesenchymal transition is a determinant of sensitivity of non-small-cell lung carcinoma cell lines and xenografts to epidermal growth factor receptor inhibition. Cancer Res 2005; 65:9455-9462.
23. Li T, Ling Y H, Goldman I D et al. Schedule-dependent cytotoxic synergism of pemetrexed and erlotinib in human non-small cell lung cancer cells. Clin Cancer Res 2007; 13:3413-3422.
24. Yauch R L, Januario T, Eberhard D A et al. Epithelial versus mesenchymal phenotype determines in vitro sensitivity and predicts clinical activity of erlotinib in lung cancer patients. Clin Cancer Res 2005; 11:8686-8698.
25. Perez-Soler R, Chachoua A, Hammond L A et al. Determinants of tumor response and survival with erlotinib in patients with non-small-cell lung cancer. J Clin Oncol 2004; 22:3238-3247.
26. Bonomi P D, Buckingham L, Coon J Selecting patients for treatment with epidermal growth factor tyrosine kinase inhibitors. Clin Cancer Res 2007;13:s4606-s4612.
27. Sequist L V, Bell D W, Lynch T J et al. Molecular predictors of response to epidermal growth factor receptor antagonists in non-small-cell lung cancer. J Clin Oncol 2007; 25:587-595.
28. Ciardiello F, Tortora G. A novel approach in the treatment of cancer: targeting the epidermal growth factor receptor. Clin Cancer Res 2001;7: 2958-70.
29. Klapper L N, Kirschbaum M H, Sela M, Yarden Y. Biochemical and clinical implications of the ErbB/HER signaling network of growth factor receptors. Adv Cancer Res 2000;77: 25-79.
30. Salomon D S, Brandt R, Ciardiello F, Normanno N. Epidermal growth factor-related peptides and their receptors in human malignancies. Crit. Rev Oncol Hematol 1995;19: 183-232.
31. Mayer A, Takimoto M, Fritz E, Schellander G, Kofler K, Ludwig H. The prognostic significance of proliferating cell nuclear antigen, epidermal growth factor receptor, and mdr gene expression in colorectal cancer. Cancer 1993;71: 2454-60.
32. Mendelsohn J. Blockade of receptors for growth factors: an anticancer therapy—the fourth annual Joseph H Burchenal American Association of Cancer Research Clinical Research Award Lecture. Clin Cancer Res 2000;6: 747-53.
33. Yamatodani T, Ekblad L, Kjellen E, Johnsson A, Mineta H, Wennerberg J. Epidermal growth factor receptor status and persistent activation of Akt and p44/42 MAPK pathways correlate with the effect of cetuximab in head and neck and colon cancer cell lines. J Cancer Res Clin Oncol 2009;135: 395-402.
34. Chung K Y, Shia J, Kemeny N E, et al. Cetuximab shows activity in colorectal cancer patients with tumors that do not express the epidermal growth factor receptor by immunohistochemistry. J Clin Oncol 2005;23: 1803-10.
35. Cunningham D, Humblet Y, Siena S, et al. Cetuximab monotherapy and cetuximab plus irinotecan in irinotecan-refractory metastatic colorectal cancer. N Engl Med 2004;351: 337-45.
36. Saltz L B, Meropol N J, Loehrer P J, Sr., Needle M N, Kopit J, Mayer R J. Phase II trial of cetuximab in patients with refractory colorectal cancer that expresses the epidermal growth factor receptor. J Clin Oncol 2004;22: 1201-8.
37. Lenz H J, Mayer R J, Gold P J, et al. Activity of cetuximab in patients with colorectal cancer refractory to both irinotecan and oxaliplatin. J Clin Oncol (Meeting Abstracts) 2004;22: 3510-.
38. Schrag D. The price tag on progress—chemotherapy for colorectal cancer. N Engl Med 2004;351: 317-9.
39. Siena S, Sartore-Bianchi A, Di Nicolantonio F, Balfour J, Bardelli A. Biomarkers predicting clinical outcome of epidermal growth factor receptor-targeted therapy in metastatic colorectal cancer. J Natl Cancer Inst 2009;101: 1308-24.
40. Rice S D, Bush J E, Brower S L. Assessment of erlotinib in chemoresponse assay. Anticancer Res 2009;29: 1993-7.
41. Raben D, Helfrich B, Chan D C, et al. The effects of cetuximab alone and in combination with radiation and/or chemotherapy in lung cancer. Clin Cancer Res 2005;11: 795-805.
42. Janmaat M L, Kruyt F A, Rodriguez J A, Giaccone G. Response to epidermal growth factor receptor inhibitors in non-small cell lung cancer cells: limited antiproliferative effects and absence of apoptosis associated with persistent activity of extracellular signal-regulated kinase or Akt kinase pathways. Clin Cancer Res 2003;9: 2316-26.
43. Mukohara T, Engelman J A, Hanna N H, et al. Differential effects of gefitinib and cetuximab on non-small-cell lung cancers bearing epidermal growth factor receptor mutations. J Natl Cancer Inst 2005;97: 1185-94.
44. Lynch T J, Lilenbaum R, Bonomi P, et al. A phase II trial of cetuximab as therapy for recurrent non-small cell lung cancer (NSCLC). J Clin Oncol (Meeting Abstracts) 2004;22: 7084.
45. Hanna N, Lilenbaum R, Ansari R, et al. Phase II trial of cetuximab in patients with previously treated non-small-cell lung cancer. J Clin Oncol 2006;24: 5253-8.
46. Ademuyiwa F O, Hanna N. Cetuximab in non-small cell lung cancer. Expert Opin Biol Ther 2008;8: 107-13.
47. Martinelli E, De Palma R, Orditura M, De Vita F, Ciardiello F. Anti-epidermal growth factor receptor monoclonal antibodies in cancer therapy. Clin Exp Immunol 2009;158: 1-9.
48. Khambata-Ford S, Garrett C R, Meropol N J, et al. Expression of epiregulin and amphiregulin and K-ras mutation status predict disease control in metastatic colorectal cancer patients treated with cetuximab. J Clin Oncol 2007;25: 3230-7.

1. A method for predicting a patient's response to a monoclonal epidermal growth factor receptor (EGFR) antibody, comprising:
culturing malignant cells from said patient;
contacting the cultured cells with a monoclonal EGFR antibody, and evaluating the cultured cells for a cytotoxic response, wherein the cytotoxic response is indicative of the patient's response to the monoclonal EGFR antibody.

2. The method of claim 1, wherein the monoclonal EGFR antibody is a chimeric monoclonal antibody.

3. The method of claim 2, wherein the monoclonal EGFR antibody is cetuximab or panitumumab.

4. The method of claim 3 wherein the monoclonal EGFR antibody is cetiximab.

5. The method of claim 1, wherein the patient has lung cancer.

6. The method of claim 5, wherein the patient has non-small cell lung cancer (NSCLC).

9. The method of claim 1, wherein the patient has colorectal cancer.

10. The method of claim 1, wherein the patient has head and neck cancer.

11. The method of claim 1, wherein the patient has breast cancer.

12. The method of any one of claims 1 to 9, wherein the patient has previously received a first line of chemotherapy.

13. The method of any one of claims 1 to 10, wherein the patient is a non-smoker.

14. The method of claim 11, wherein the patient has never been a smoker.

15. The method of claim 1, wherein the patient has pancreatic cancer.

16. The method of any of claims 1 to 13, wherein the cultured cells are enriched for malignant cells.

15. The method of claim 14, wherein the cultured cells are from monolayers grown from multicellular particulates of tumor tissue.

16. The method of claim 15, wherein the multicellular particulates are prepared by mincing the tumor tissue.

17. The method of claim 15 or 16, wherein the multicellular particulates are agitated to release malignant cells, and/or the multicellular particulates are removed from the monolayer at about 20% to about 70% confluency.

18. The method of any one of claims 17 to 19, wherein the multicellular particulates have a size of from about 0.25 to about 1.5 mm³.

19. The method of any of claims 17 to 20, wherein the multicellular particulates have smooth cut edges.

20. The method of any of claims 1 to 19, wherein the malignant cells are contacted with a range of doses of said monoclonal EGFR antibody.

21. The method of claim 20, further comprising preparing a dose response curve for said monoclonal EGFR antibody.

22. The method of any one of claims 1 to 21, further comprising, indicating whether said patient will be responsive, non-responsive, or intermediately responsive to said monoclonal EGFR antibody, or indicating whether said cultured cells were responsive, non-responsive, or intermediately responsive to said monoclonal EGFR antibody.
